(19) United States
(12) Patent Application Publication
(10) Pub. No.: US 2011/0045055 A1
(43) Pub. Date: Feb. 24, 2011

(54) MEDICAL DEVICES CONTAINING THERAPEUTIC AGENTS

(75) Inventors: John Hingston, County Galway (IE); Michael Robichaud, Galway (IE); Jan Weber, Maastricht (NL); Adrian McNamara, Galway (IE); Aiden Flanagan, Galway (IE); Fergal Horgan, County Mayo (IE)

Correspondence Address: MAYER & WILLIAMS PC, 251 NORTH AVENUE WEST, Suite 201, WESTFIELD, NJ, 07090, US

(73) Assignee: BOSTON SCIENTIFIC SCIMED, INC., Maple Grove, MN (US)

(21) Appl. No.: 12/860,304

(22) Filed: Aug. 20, 2010

Related U.S. Application Data
(60) Provisional application No. 61/235,958, filed on Aug. 21, 2009.

(51) Int. Cl. A61F 2/82; A61M 31/00; A61P 9/08

(52) U.S. Cl. 424/424; 424/422

(57) ABSTRACT

According to one aspect of the invention, implantable or insertable medical devices are provided which can delay release of one or more therapeutic agents for a predetermined time after implantation in a subject. In various embodiments, a therapeutic agent delivery profile of this type is provided by employing a temporary barrier layer which initially permits little to no release of the therapeutic agent, but which layer permits much greater release levels after a predetermined period of time. Other aspects of the invention relate to methods of forming such devices and to methods of using such devices.

### RELATED APPLICATION

This application claims priority from U.S. provisional application 61/235,958, filed Aug. 21, 2009, which is incorporated by reference herein in its entirety.

### TECHNICAL FIELD

This invention relates to medical devices that release therapeutic agents.

### BACKGROUND OF THE INVENTION

The in-situ delivery of therapeutic agents within the body of a patient is common in the practice of modern medicine. In-situ delivery of therapeutic agents is often implemented using medical devices that may be temporarily or permanently placed at a target site within the body. These medical devices can be maintained, as required, at their target sites for short or prolonged periods of time, in order to deliver therapeutic agents to the target site.

For example, in recent years, drug eluting coronary stents, which are commercially available from Boston Scientific Corp. (TAXUS and PROMUS), Johnson & Johnson (CYPHER) and others, have been widely used for maintaining vessel patency after balloon angioplasty. These products are based on metallic expandable stents with polymer coatings that release anti-proliferative drugs at a controlled rate and total dose.

### SUMMARY OF THE INVENTION

According to one aspect of the invention, implantable or insertable medical devices are provided which can delay release of one or more therapeutic agents for a predetermined time after implantation in a subject. In various embodiments, a therapeutic agent delivery profile of this type is provided by employing a temporary barrier layer which initially permits little to no release of the therapeutic agent, but which layer permits much greater release levels after a predetermined period of time.

Other aspects of the invention relate to methods of forming such devices and to methods of using such devices.

An advantage of certain embodiments of the present invention is that medical devices may be provided, in which the release of one or more therapeutic agents is controlled and tailored.

An advantage of certain other embodiments of the present invention is that medical devices may be provided, which are quickly encapsulated with endothelial cells but which do not result in significant restenosis due to excessive cell growth after implantation.

These and many other aspects, embodiments and advantages of the present invention will become immediately apparent to those of ordinary skill in the art upon review of the Detailed Description and Claims to follow.

### BRIEF DESCRIPTION OF THE DRAWINGS

FIGS. 1, 2, 3, 4, 5, 6, 6A, 7, 8, 9 and 9A are schematic partial cross-sections of medical devices, in accordance with various embodiments of the present invention.

FIGS. 10A-10G are schematic partial cross-sections illustrating the formation of a medical device, in accordance with an embodiment of the present invention.

FIG. 11 is a schematic representation of a release profile from a medical device in accordance with the prior art.

FIG. 12 is a hypothetical release profile from a medical device in accordance with an embodiment of the present invention.

FIG. 13 is a hypothetical release profile from a medical device in accordance with another embodiment of the present invention.

### DETAILED DESCRIPTION

According to one aspect, the present invention is directed to medical devices which can delay release of one or more therapeutic agents for a predetermined time after implantation in a subject. One way of providing a therapeutic agent delivery profile of this type is to employ a temporary barrier layer which initially permits little to no release of the therapeutic agent, but which layer permits much greater release levels after a desired period of time (e.g., because the barrier layer increases in permeability over time, because the barrier layer ruptures after a period of time, etc.), thereby allowing for therapeutic agent release to begin or to increase substantially. In other words, a temporary barrier layer is employed which delays release of the therapeutic agent.

“Therapeutic agents,” “drugs,” “biologically active agents,” “pharmaceutically active agents,” and other related terms may be used interchangeably herein.

Examples of medical devices benefiting from the present invention vary widely and include implantable or insertable medical devices, for example, stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, catheters (e.g., urological catheters or vascular catheters such as balloon catheters and various central venous catheters), guide wires, balloons, filters (e.g., vena cava filters and mesh filters for distil protection devices), embolization devices including cerebral aneurysm filler coils (including Guglielmi detachable coils and metal coils), septal defect closure devices, drug depots that are adapted for placement in an artery for treatment of the portion of the artery distal to the device, myocardial plugs, patches, pacemakers, leads including pacemaker leads, defibrillation leads and coils, ventricular assist devices including left ventricular assist hearts and pumps, total artificial hearts, shunts, valves including heart valves and vascular valves, anastomosis clips and rings, cochlear implants, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, artificial ligaments, tacks for ligament attachment and meniscal repair, joint prostheses, spinal discs and nuclei, orthopedic prosthesis such as bone grafts, bone plates, fins and fusion devices, orthopedic fixation devices such as interference screws in the ankle, knee, and hand areas, rods and pins for fracture fixation, screws and plates for craniomaxillofacial repair, dental implants, or other devices that are implanted or inserted into the body and from which therapeutic agent is released.

The medical devices of the present invention include, for example, implantable and insertable medical devices that are used for systemic treatment or diagnosis, as well as those that are used for the localized treatment or diagnosis of any mammalian tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes, ears, spine, nervous system, brain, lungs, trachea, esophagus, intestines, stomach, liver and pancreas, skeletal muscle, smooth muscle, breast, dermal tissue, cartilage, tooth and bone.

As used herein, “treatment” refers to the prevention of a disease or condition, the reduction or elimination of symptoms associated with a disease or condition, or the substantial or complete elimination of a disease or condition. Subjects are vertebrate subjects, more typically mammalian subjects including human subjects, pets and livestock.

As noted above, the present invention is directed to medical devices which can delay release of one or more therapeutic agents for a predetermined time after implantation in a subject, for example, through the use of a layer that initially acts as a temporary barrier to therapeutic agent release (thereby permitting little to no release of the therapeutic agent during the predetermined time period), after which time the layer allows for therapeutic agent release to begin or increase dramatically.

Typically, medical devices in accordance with the invention comprise a substrate and one or more therapeutic-agent-containing regions from which release is controlled via one or more temporary barrier layers.

Materials for forming substrates and temporary barrier layers include biodisintegrable materials and biostable materials.

As used herein, a “biodisintegrable” material is one that, upon placement at an implantation/insertion site in the body, is dissolved, biodegraded, resorbed, and/or otherwise substantially removed from the placement site during the anticipated placement period for the device, up to the lifetime of the subject (e.g., from 90 to 95 to 97 to 99 to 100 wt % removed). Typically, the material is substantially removed in vivo in a period of two years or less. In certain embodiments, the material is substantially removed over a period of days (e.g., within 5 days). For example, in case where a biodisintegrable temporary barrier layer is employed, the biodisintegration time can be used to dictate the initial period of time during which release of the therapeutic agent is prevented.

As used herein, a “biostable” material is one that, upon placement at an implantation/insertion site in the body, remains substantially intact over the anticipated placement period for the device (up to the lifetime of the subject).

Biostable and biodisintegrable material for use in the devices of the invention include (a) organic materials (i.e., materials containing organic species, typically 50 wt % or more, for example, from 50 wt % to 75 wt % to 90 wt % to 95 wt % to 97.5 wt % to 99 wt % or more) such as polymeric materials (i.e., materials containing polymers, typically 50 wt % or more polymers, for example, from 50 wt % to 75 wt % to 90 wt % to 95 wt % to 97.5 wt % to 99 wt % or more) and biologics, (b) inorganic materials (i.e., materials containing inorganic species, typically 50 wt % or more, for example, from 50 wt % to 75 wt % to 90 wt % to 95 wt % to 97.5 wt % to 99 wt % or more), such as metallic materials (i.e., materials containing metals, typically 50 wt % or more, for example, from 50 wt % to 75 wt % to 90 wt % to 95 wt % to 97.5 wt % to 99 wt % or more) and non-metallic inorganic materials (i.e., materials containing non-metallic inorganic materials, typically 50 wt % or more, for example, from 50 wt % to 75 wt % to 90 wt % to 95 wt % to 97.5 wt % to 99 wt % or more) (e.g., carbon, semiconductors, glasses and ceramics, which may contain various metal- and non-metal-oxides, various metal- and non-metal-nitrides, various metal- and non-metal-carbides, various metal- and non-metal-borides, various metal- and non-metal-phosphates, and various metal- and non-metal-sulfides, among others), and (c) hybrid materials (e.g., hybrid organic-inorganic materials, for instance, polymer/metallic inorganic and polymer/non-metallic inorganic hybrids).

Specific examples of inorganic non-metallic materials may be selected, for example, from materials containing one or more of the following: metal oxide ceramics, including aluminum oxides and transition metal oxides (e.g., oxides of titanium, zirconium, hafnium, tantalum, molybdenum, tungsten, rhenium, iron, niobium, iridium, etc.); silicon; silicon-based ceramics, such as those containing silicon nitrides, silicon carbides and silicon oxides (sometimes referred to as glass ceramics); calcium phosphate ceramics (e.g., hydroxyapatite); carbon; and carbon-based ceramic-like materials such as carbon nitrides.

Specific examples of metallic materials may be selected, for example, from metals such as gold, iron, niobium, platinum, palladium, iridium, osmium, rhodium, titanium, tantalum, tungsten, ruthenium, and magnesium, among others, and alloys such as those comprising iron and chromium (e.g., stainless steels, including platinum-enriched radiopaque stainless steel), niobium alloys, titanium alloys, alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N), alloys comprising cobalt, chromium, tungsten and nickel (e.g., L605), alloys comprising nickel and chromium (e.g., inconel alloys), and biodisintegrable alloys including alloys of magnesium, zinc and/or iron (including their alloys with combinations of each other and Ce, Ca, Zr, Li, etc., for example, alloys containing magnesium and one or more of Fe, Ce, Ca, Zn, Zr and Li, alloys containing iron and one or more of Mg, Ce, Ca, Zn, Zr and Li, alloys containing zinc and one or more of Fe, Mg, Ce, Ca, Zr and Li, etc.), among others.

Specific examples of organic materials include polymers (biostable or biodisintegrable) and other high molecular weight organic materials, and may be selected, for example, from suitable materials containing one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, and glycosaminoglycans such as hyaluronic acid; as well as blends and further copolymers of the above.

As noted above, in addition to a substrate, medical devices in accordance with the invention typically contain one or more therapeutic-agent-containing regions from which release is controlled via one or more temporary barrier layers.

The therapeutic-agent-containing regions may contain, for example, from 1 wt % or less to 2 wt % to 5 wt % to 10 wt % to 25 wt % to 50 wt % to 75 wt % to 90 wt % to 95 wt % to 97.5 wt % to 99 wt % to 100 wt % of one or more therapeutic agents. Examples of materials other than therapeutic agents which can be used to form the therapeutic-agent-containing regions include materials that serve as binders, matrices, diluents, fillers, etc. for the therapeutic agent (collectively referred to herein as “excipients”). Examples of such materials may be selected, for example, from suitable members of the organic, inorganic and organic-inorganic hybrid materials listed above, among others. In various embodiments, the therapeutic agent containing regions are substantially pure (defined herein as containing 95 wt % or more of a given therapeutic agent).

Several embodiments of the invention will now be described below. Although various embodiments are described which use vascular stents as specific illustrative medical devices, the invention is clearly not so-limited.

With regard to vascular stents, when a bare metal stent is implanted into a subject, various cells, including smooth muscle cells and endothelial cells, typically grow to encapsulate the stent. Sometimes these cells (e.g., smooth muscle cells) grow excessively, creating a new narrowing (restenosis) after a period of time. Although excessive cell growth is unwanted, a certain amount of cell growth is actually healthy and desirable, for example, where the endothelial cells proliferate and grow over the stent struts to form a confluent layer of endothelial cells. In this regard, formation of a functional endothelial cell layer has been reported to be an effective way to reduce or eliminate inflammation and thrombosis, which are sometimes associated with implantable devices. See, e.g., J. M. Caves et al., J. Vasc. Surg. (2006) 44: 1363-1368.

Commercially available drug eluting stents typically begin eluting a drug that targets excessive cell growth (e.g., smooth muscle cell growth) immediately upon implantation. Consequently, such stents have been found to decrease the incidence of restenosis in subjects. Unfortunately, various drugs that can reduce undesirable excessive cell growth can also lead to a reduction in the growth of beneficial cells, including endothelial cells. In other words, when such stents are implanted, cell growth is reduced immediately, avoiding excessive undesirable cell growth, while potentially having a negative side-effect as well on the encapsulation of the stent and the development of a functional endothelial cell layer.

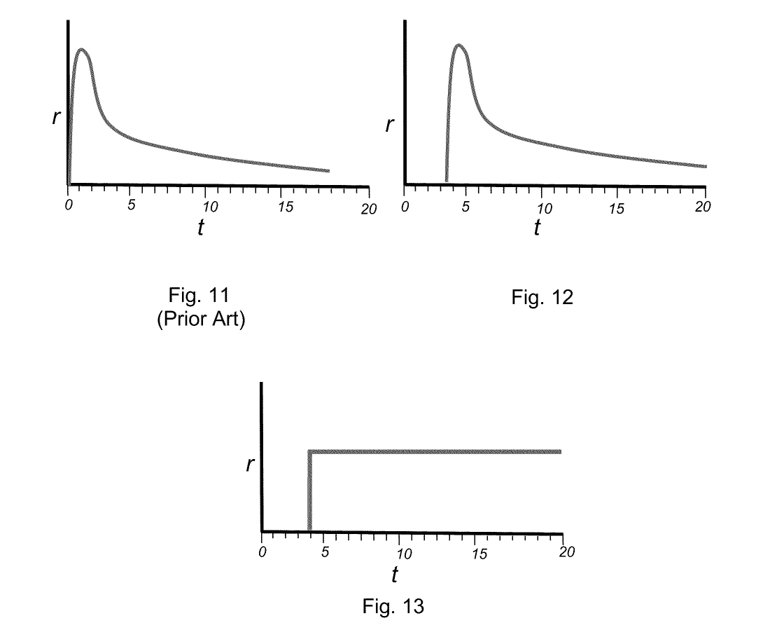

For purposes of illustration, a schematic representation of a release profile from such a device is shown in FIG. 11 wherein t is the implantation time (in arbitrary units) and r is the release rate (in arbitrary units). In FIG. 11, drug release begins essentially immediately upon implantation at time t=0, reaching a maximum rate at time period t=2 after which the release rate decays, asymptotically approaching a zero release rate.

In accordance with the present invention, on the other hand, one may take a hybrid approach by providing an initial period during which there is little to no release of a drug that targets excessive cell growth. This initial period, which may be within the range of, for example, from 5 to 28 days (e.g., from 5 to 10 to 14 to 21 to 28 days), among other possibilities, allows for cell growth to proceed unchecked in the early stages of implantation, for example, allowing for partial or complete stent strut encapsulation. This initial period is then followed by a period in which a drug is released in amounts that are effective to control excessive cell growth (e.g., excessive smooth muscle cell growth that can lead to restenosis).

For illustration purposes, a hypothetical release profile from such a device is shown in FIG. 12 wherein, as in FIG. 11, t is the implantation time (arbitrary units) and r is the release rate (arbitrary units). Unlike FIG. 11, however, FIG. 12 shows a drug release profile wherein drug release does not commence until the device is implanted for a predetermined period of time (t=3), reaching a maximum rate at time t=5, after which the release rate decays. The predetermined period of time may be within the range of, for example, from 5 to 28 days as indicated above, among other possibilities.

The release profiles in FIGS. 11 and 12 illustrate an initial burst followed by an exponential decay of the release rate. However, various other release profiles are clearly possible. For example, FIG. 13 shows drug release profile wherein drug release does not commence until the device is implanted for a predetermined period of time (t=3) (e.g., 5 to 28 days, among other possibilities), at which point drug is released at a constant rate (until the drug is gone). A profile where the release rate is substantially constant with time is sometimes called a zero order release profile.

In certain embodiments of the invention, additional drugs other than anti-restenotic drugs may be released during the initial period time (e.g., between t=0 and t=3 in FIG. 12 and between t=0 and t=4 in FIG. 13). For example, during this period it may be desirable to release additional drugs such as antithrombotic agents or agents which promote the attachment and/proliferation of endothelial cells without promoting growth of other types of cells (e.g., smooth muscle cells).

One way of providing a device with a delayed drug release profile is to employ a temporary barrier layer which initially prevents drug release (e.g., is impermeable/impenetrable to the drug) but which permits drug release (e.g., becomes permeable/penetrable) after a desired period of time. As indicated above, temporary barrier layers may be formed, for example, from a suitable organic, inorganic or organic-inorganic hybrid material.

In certain embodiments of the invention, a temporary barrier layer is provided which creates a release profile in which the cumulative release of therapeutic agent that occurs over the first several days of implantation (e.g., for a period ranging from the first 5 to 28 days of implantation) is less than the minimum therapeutic level. For example, the cumulative release during this period may be less than 10% of the total cumulative release (e.g., less than 10%, less than 5% or even less than 1%) that occurs over the normal lifetime of the device (e.g., one year or more) in certain embodiments.

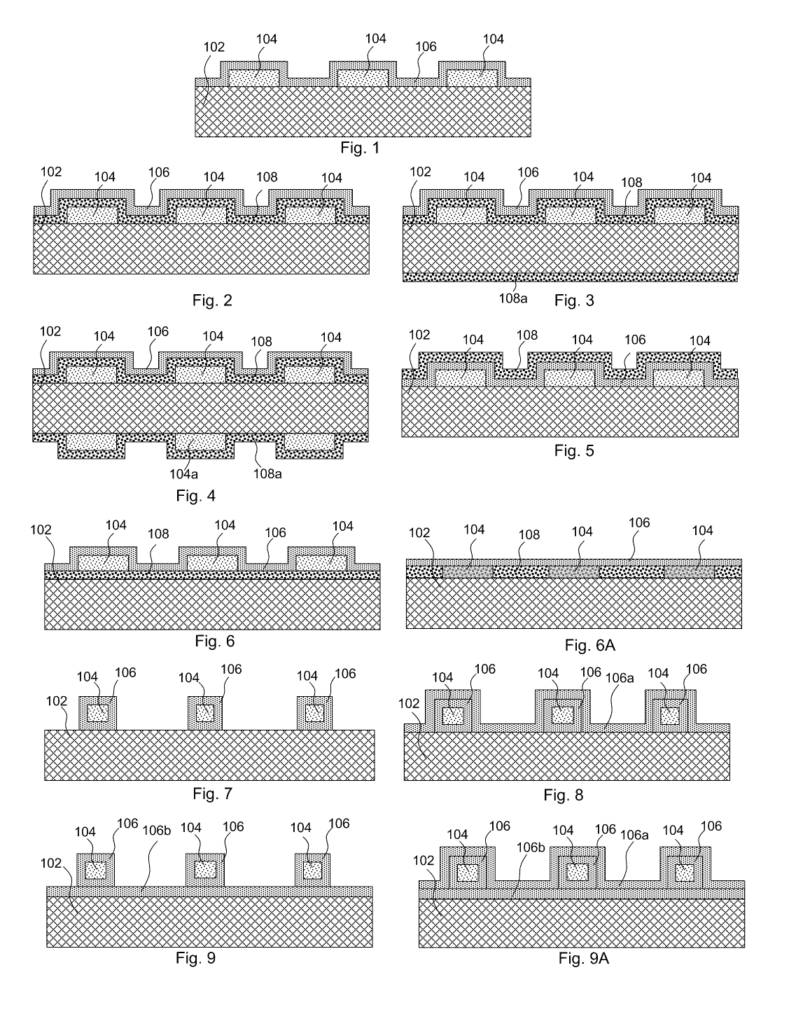

Referring to FIG. 1, there is shown in cross-section a medical device substrate 102 (e.g., a stainless steel stent strut, etc.), having disposed on its surface (e.g., its outer, vessel-wall-contacting surface, also known as its abluminal surface) various therapeutic-agent-containing regions 104 (e.g., antirestenotic-agent-containing regions).

In some embodiments, the therapeutic-agent-containing regions 104 may have a width, and in some embodiments have a length and a width, that are each less than 100 μm, preferably less than 50 μm. In certain of these embodiments, many regions of therapeutic-agent-containing material are formed, for example, >100, >1000, or more regions per mm².

Disposed over the substrate 102 and the therapeutic-agent-containing regions 104 is a temporary barrier layer 106 that initially prevents release of the therapeutic agent but permits release after a predetermined period of time. The temporary barrier layer may be, for example, a biodisintegrable organic material such as a sugar, polysaccharide or polypeptide (e.g., sucrose, lactose, heparin, albumin, etc.) or synthetic polymer (e.g., a biodegradable polymer such as polymers of lactide, glycolide, lactide/glycolide, caprolactone, etc.) or a biodisintegrable inorganic material such as a biodisintegrable metal (e.g., magnesium, magnesium alloy, zinc, zinc alloy, etc.), a biodisintegrable metal- or semi-metal-oxides, phosphates or sulfates (e.g., alumina, silica, hydroxyapatite, zirconia, titanium oxide, platinum oxide, calcium oxide, magnetite, etc.) or a biodisintegrable metal halide (e.g., magnesium fluoride, magnesium chloride, etc.), among many other possibilities.

Individual therapeutic-agent-containing regions 104 may be formed using a number of techniques. For example, such regions may be formed by depositing a therapeutic-agent-containing solution, dispersion or melt (which may contain additional excipients as noted above) onto a substrate surface 102 using a suitable application technique such as ink jet droplet deposition, micro-contact printing, nanopipetting, dip pen nanolithography, selective roll coating and spray coating (in which parameters are controlled to achieve deposition of individual particles or in which masking is used for selective deposition), among others. Such regions may also be formed by manual placement, for example, by application of therapeutic-agent-containing regions 104 (e.g., cubes, spheres, etc.) after applying a suitable adhesive to the surface), among other methods.

The substrate surface 102 in FIG. 1 comprises a first area which is covered by the therapeutic-agent-containing regions 104 and a second area which is not covered by the therapeutic-agent-containing regions 104. For example, the first area may preferably comprise between 10% and 90% (more preferably between 25% and 75%) of the total abluminal area of the stent surface and the second area may comprise the remainder of the abluminal area. Such embodiments may be desirable, for instance, where it is desirable to have contact between the temporary barrier layer 106 and the substrate 102 in some places to enhance adhesion of the temporary barrier layer 106 to the device.

The temporary barrier layer 106 may be formed, for example, using a suitable deposition technique, for example, ink jet droplet deposition, micro-contact printing, nanopipetting, dip pen nanolithography, roll coating or spray coating for organic barrier layers or a suitable vapor deposition technique such as atomic layer deposition (ALD), chemical vapor deposition (CVD) or physical vapor deposition (PVD) for inorganic barrier layers, among others. In various embodiments, temporary barrier layer 106 is formed from a biodisintegrable material, in which case the thickness of the temporary barrier layer 106 will generally be a function, for example, of the biodisintegration rate of the material forming the layer 106 and of the delay in release that is desired.

For instance, in some embodiments, a temporary barrier layer 106 may be formed using atomic layer deposition (ALD) technology. Such technology is suitable for forming a number of types of layers including, for example, oxides (e.g. Al₂O₃, TiO₂, Ta₂O₅, Nb₂O₅, ZrO₂, HfO₂, ZnO, MgO, La₂O₃, Y₂O₃, CeO₂, Sc₂O₃, B₂O₃, CO₂O₃, CuO, Fe₂O₃, NiO, Ga₂O₃, WO₃, etc.), metals (e.g., Pt, Ru, Ir, Pd, Cu, Fe, Co, Ni, W, etc.), nitrides (AlN, TaNx, NbN, TiN, MoN, ZrN, HfN, GaN, WxN, InN) and carbides (e.g., TiC, NbC, TaC, etc.), and metal phosphates such as hydroxyapatite [Ca₁₀(PO₄)₆(OH)₂].

ALD is a surface controlled layer-by-layer (LbL) process, which is capable of depositing conformal, ultrathin, high purity, pin-hole free layers on a given substrate. X. Liang et al., J. Am. Ceram. Soc., 90(1), 2007, 57-63. In ALD, a substrate is exposed to alternating gaseous precursors which combine to form the coating material of interest. The film thickness is controlled by the number of times the substrate is exposed to the alternating gases.

ALD is based on a sequence of two self-limiting reactions between gas phase precursor molecules and a solid surface. During the reaction sequence, only one reactant is present in the reaction zone at a time, preventing unwanted gas phase reactions. Because only a finite number of reactive sites exist on the surface, reactions of the precursors are inherently self-limiting. Moreover, since gas phase reactants are utilized, ALD does not require line-of-sight, allowing conformal coatings to be readily created. C. F. Herrmann et al., Applied Physics Letters, 87, 123110 (2005)

For example, using ALD, various metals and non-metals can be deposited at relatively low temperatures, including Al₂O₃/alumina (see, e.g., X. Liang et al., J. Am. Ceram. Soc., 90(1), 2007, 57-63, who describe a process whereby alumina is deposited by repeated exposure to trimethylaluminum and H₂O vapor at 77° C. in a repeated alternating sequence), TiO₂/titania (see, e.g., Jae-Hwang Lee et al., Applied Physics Letters 90 151101 (2007) who describe ALD deposition of TiO₂ at 100° C. from TiCl₄ and H₂O) and SiO₂/silica (see, e.g., J. W. Klaus et al., Surface Review and Letters, Vol. 6, Nos. 3 & 4 (1999) 435-448, who describe ALD deposition of SiO₂ at temperatures as low as 300K from SiCl₄ and H₂O using pyridine as a catalyst; see also U.S. Pat. No. 7,077,904 to Cho et al.), among many others, for use in the invention.

In other embodiments, temporary barrier layers 106 may be formed using physical layer deposition (PVD) methods. Some specific PVD methods that may be used to form temporary barrier layers 106 in accordance with the present invention include evaporation, sublimation, sputter deposition and laser ablation deposition. Examples of biodisintegrable inorganic materials that may be formed using PVD include various metals, metal oxides and metal halides.

In some instances, layer properties may be improved by conducting thin film deposition (e.g., by evaporation, sublimation, sputtering, laser ablation, or another type of PVD) while simultaneously performing directed ion bombardment of the film surface from an ion source. Such techniques are referred to as ion beam-assisted deposition (IBAD) techniques and can produce coatings with improved substrate adhesion, increased mass densities and decreased residual stresses. For example, L. Dumas et al., Thin Solid Films 382, (2001) 61-68 described a process whereby various magnesium fluoride thin films are deposited by electron beam evaporation of MgF₂ with simultaneous argon ion bombardment (max. temp. 70° C.).

In other embodiments, temporary barrier layers 106 may be formed using chemical vapor deposition (CVD) technology. Such technology is suitable for forming a number of types of layers including, for example, various metal oxide layers. In this regard, see, e.g., M. Seman et al., Applied Physics Letters 90, 131504 (2007) who describe plasma-enhanced CVD of Ta₂O₅ at temperatures as low as 90° C. from pentaethoxy tantalum and O₂.

In the above described embodiments, release is controlled by selection of a suitable temporary barrier layer 106.

Release can be further controlled by providing the devices of the invention with a porous layer that is disposed between a source of therapeutic agent and the exterior of the device. Porous layers for the devices of the present invention include nanoporous, microporous, mesoporous, and macroporous layers. As used herein a “porous” layer is a layer that contains pores. A “nanoporous layer” is a layer that contains nanopores. A “microporous layer” is a layer that contains micropores. A “mesoporous layer” is a layer that contains mesopores. A “macroporous layer” is a layer that contains macropores. In accordance with the International Union of Pure and Applied Chemistry (IUPAC), a “nanopore” is a pore having a width that does not exceed 50 nm (e.g., from 0.5 nm or less to 1 nm to 2.5 nm to 5 nm to 10 nm to 25 nm to 50 nm), and this definition is used herein. As used herein, nanopores include “micropores,” which are pores having a width that does not exceed 2 nm, and “mesopores,” which are range from 2 to 50 nm in width. As used herein, “macropores” are larger than 50 nm in width and are thus not nanopores.

For example, FIG. 2 shows a medical device substrate 102 (e.g., a stent strut), having disposed on its surface various therapeutic-agent-containing regions 104 (e.g., antirestenotic-agent-containing regions). Disposed over the substrate 102 and therapeutic-agent-containing regions 104 is a porous layer 108, for example, a nanoporous layer with a sufficiently small pore size to substantially regulate the release of the drug (e.g., allowing for zero order release, among other profiles). Porous layers may also be desirable in that they are known to support tissue growth (along with other types of textured surfaces). Disposed over the porous layer 108 is a temporary barrier layer 106 that initially prevents release of the therapeutic agent through the porous layer 108, but which permits the same after a predetermined period of time (e.g., a period of 5 to 28 days after implantation due to biodisintegration of the temporary barrier layer 106). The porous layer 108, on the other hand, may be formed using a biostable inorganic material (e.g., iridium, tantalum, etc.) or a biodisintegrable inorganic material that biodisintegrates at a slower rate than that of the overlying temporary barrier layer 106. For example, in the case where the substrate 102 is a stent strut, the therapeutic-agent-containing regions 104, porous layer 108 and temporary barrier layer 106 may be formed on the abluminal surface of the stent strut (the surface of the stent strut that faces the vessel wall upon implantation), where the porous layer 108 and temporary barrier layer 106 act to regulate release of an antirestenotic agent from the therapeutic-agent-containing regions 104.

Nanoporous metallic and ceramic layers for use in structures such as those of FIG. 2 can be formed, for example, by accelerating charged nanoparticles into a substrate. See, e.g., WO 2008/140482 to Weber et al. As a specific example, a system for performing nanoparticle deposition along the lines described above is available from Mantis Deposition Ltd., Thame, Oxfordshire, United Kingdom, who market a high-pressure magnetron sputtering source which is able to generate nanoparticles from a sputter target with as few as 30 atoms up to those with diameters exceeding 15 nm. (A system similar to the Mantis system can be obtained from Oxford Applied Research, Witney, Oxon, UK.) This system is operated at about 5×10⁻⁵ mbar, although the precise operating pressure used will vary widely, depending on the specific process and system that is employed, among other factors. The size of the nanoparticles is affected by several parameters, including the nanoparticle material, the distance between the magnetron surface and the exit aperture (e.g., larger distances have been observed to create larger nanoparticles), gas flow (e.g., higher gas flows have been observed to create smaller nanoparticle sizes), and gas type (e.g., helium has been observed to produce smaller particles than argon). For a particular setting, the size distribution can be measured using a linear quadrapole device placed after the exit aperture of the magnetron chamber. The quadrapole device can also be used in-line to select a narrow nanoparticle size range for deposition. Systems like the Mantis Deposition Ltd. system can produce nanoparticles, a large fraction of which of which (approximately 40% to 80%) have a charge of one electron. Consequently, a magnetic field or a secondary electric field can be used to separate particles of similar weight from one another (because lighter particles are deflected to a greater degree in a given field than are the larger particles of the same charge). For example, the above Mantis Deposition Ltd. system is able to produce charged nanoparticle streams with a very narrow mass distribution. Moreover, it is possible to accelerate the negatively charged particles onto a positively biased surface in order to impact the particles on the surface with elevated kinetic energy. A positively biased grid may also be used to accelerate the particles, allowing the particles to pass through holes in the grid and impinge on the surface. By altering the bias voltage from low to high values the deposited film changes from porous loosely bound nanoparticles to a solid film of metal. Due to the fact that the amount of energy needed to melt the individual nanoparticles is relatively low compared to the energy needed to increase the bulk temperature of an underlying structure, this process is effectively performed at or near room temperature. When using a system like the Mantis Deposition Ltd. system, it has been found that the bias voltage (which may vary, for example, from 10 V to 5000 V) and the particle size (which may vary, for example, from 0.7 nm to 25 nm) has a significant effect upon drug release, with higher voltages and smaller particle sizes yielding coatings with reduced drug release.

FIG. 5 is like FIG. 2 in that it shows a structure that contains a medical device substrate 102, various therapeutic-agent-containing regions 104 disposed on the substrate 102, a temporary barrier layer 106 and a porous layer 108. However, in contrast to FIG. 2, the positions of the a temporary barrier layer 106 and a porous layer 108 are reversed such that the temporary barrier layer 106 is disposed over and in contact with the therapeutic-agent-containing regions 104 and a portion of the substrate 102, and the porous layer 108 is disposed over and in contact with the temporary barrier layer 106. As with the structure of FIG. 2, the temporary barrier layer 106 prevents release for a period of time after implantation. However, unlike FIG. 2, the biodisintegration of the temporary barrier layer 106 would result in loss of adhesion between the porous layer 108 and the underlying substrate 102. Such a structure may be desirable, for example, where the underlying medical device substrate is biodisintegrable, for instance, when made out of a biodisintegrable polymeric or metallic material. In such cases, local forces (e.g., local volume expansion in the case of an oxidizing metallic material, etc.) and changes in chemical conditions on the surface of the degrading substrate (e.g., a strong increase or decrease in pH) may have a destructive influence on the mechanical integrity of the porous layer, in which case it may be preferred to have no contact at all between these two elements. The presence of a porous layer around a degrading substrate may also have a positive influence on the corrosion process as it prevents the formation of a biofilm directly on the substrate which can cause a strong reduction in corrosion rate. For example, it is known that iron-based biodisintegrable stents can corrode slower than is desirable in vivo due to biofilm formation.

Turning to FIG. 3, a structure is shown which like FIG. 2 contains a medical device substrate 102, various therapeutic-agent-containing regions 104 disposed on the substrate 102, a porous layer 108 disposed over and in contact with the substrate 102 and the therapeutic-agent-containing regions 104, and a temporary barrier layer 106 disposed over and in contact with the porous layer 108. In addition, the structure shown in FIG. 3 also includes an additional porous layer 108a on the opposite side of the substrate 102, which may be included, for example to promote tissue growth. For example, in the case where the substrate 102 of FIG. 3 is a stent strut, the therapeutic-agent-containing regions 104, porous layer 108 and temporary barrier layer 106 may be formed on the abluminal surface of the stent strut (the surface of the stent strut that faces the vessel wall upon implantation), wherein the porous layer 108 and temporary barrier layer 106 act to regulate release of an antirestenotic agent in the therapeutic-agent-containing regions 104. The additional porous layer 108a may be formed on the luminal surface of the stent substrate 102 (the surface of the stent strut that faces the center of the stent through which blood flows). The pore size of the porous layer 108a may be optimized to lead to the attachment and growth of healthy endothelial cells.

In this regard, submicron topography, including pores, fibers, and elevations in the sub-100 nm range, has been observed for the basement membrane of the aortic valve endothelium as well as for other basement membrane materials. See R. G. Flemming et al., Biomaterials 20 (1999) 573-588, S. Brody et al., Tissue Eng. 2006 February; 12(2): 413-421, and S. L. Goodman et al., Biomaterials 1996; 17: 2087-95. Goodman et al. employed polymer casting to replicate the topographical features of the subendothelial extracellular matrix surface of denuded and distended blood vessels, and they found that endothelial cells grown on such materials spread faster and appeared more like cells in their native arteries than did cells grown on untextured surfaces.

Like FIG. 3, the structure shown in FIG. 4 contains a medical device substrate 102, various therapeutic-agent-containing regions 104 disposed on the substrate 102, a porous layer 108 disposed over and in contact with both the substrate 102 and the therapeutic-agent-containing regions 104, and temporary barrier layer 106 disposed over and in contact with the porous layer 108. For example, in the case where the substrate 102 is a stent strut, the therapeutic-agent-containing regions 104, porous layer 108 and temporary barrier layer 106 may be formed on the abluminal surface of the stent strut, and the porous layer 108 and temporary barrier layer 106 may act to regulate release of an antirestenotic agent from the therapeutic-agent-containing regions 104.

In addition, the structure shown in FIG. 4 also includes additional therapeutic-agent-containing regions 104a disposed on the opposite face of the substrate 102 and an additional porous layer 108a disposed over and in contact with the substrate 102 and additional therapeutic-agent-containing regions 104a. For example, in the case where the substrate 102 is a stent strut, the additional therapeutic-agent-containing regions 104a and additional porous layer 108a may be formed on the luminal surface of the stent strut 120, with an endothelial-cell-growth-promoting agent supplied in the additional therapeutic-agent-containing regions 104a. Unlike the abluminal surface, the luminal surface does not include a temporary barrier layer 106 in the embodiment shown, allowing release to begin more quickly (e.g., immediately) from the luminal surface than the abluminal surface. The porosity of the additional porous layer 108a may be optimized to regulate the release of the endothelial-cell-growth-promoting agent from the therapeutic-agent-containing regions 104a and/or optimized to enhance endothelial cell attachment and growth.

The structure shown in FIG. 6 contains a medical device substrate 102, a porous layer 108 disposed over and in contact with both the substrate 102, various therapeutic-agent-containing regions 104 disposed over and in contact with the porous layer 108, and a temporary barrier layer 106 disposed over and in contact with the therapeutic-agent-containing regions 104 and porous layer 108. Such an embodiment would provide for delayed release of the therapeutic agent from the therapeutic-agent-containing regions 104, after a delay period during which the temporary barrier layer 106 is biodisintegrated in vivo. Such an embodiment would also provide a porous surface for cell attachment and growth after this period. It is known that certain topographies stimulate cell growth and the porous layer in this case can be used to provide such a topography. See C. Wilkinson et al., “Topographical control of cells,” Biomaterials, 18 (1998) 1573-1583. This may also be useful, for example, in those cases where the barrier layer is meant to have a very short duration and where the therapeutic agent is in such a form (i.e. embedded in capsules or crystalline form) that the therapeutic agent by itself has a slow elution profile without the need for an additional barrier layer.

The structure shown in FIG. 6A is like FIG. 6 except that the therapeutic-agent-containing regions 104 are formed in the porous layer 108, for example, by applying drops of therapeutic-agent-containing solution to the porous layer 108 in spaced intervals. (Alternatively, single large therapeutic-agent-containing region may be formed in the porous layer.) Thus, the structure of FIG. 6A, includes a medical device substrate 102, a porous layer 108 disposed over and in contact with the substrate 102, therapeutic-agent-containing regions 104 formed within the porous layer 108, and a temporary barrier layer 106 disposed over and in contact with the porous layer 108 (including the therapeutic-agent-containing regions 104 formed in the porous layer 108). As with FIG. 6, such an embodiment would provide for delayed release of the therapeutic agent from the therapeutic-agent-containing regions 104 after a delay period during which the temporary barrier layer 106 is biodisintegrated in vivo. Such an embodiment would also provide a porous substrate for cell attachment and growth after this period.

In the above embodiments, the therapeutic agent is applied over a substrate 102 in the form of therapeutic-agent-containing regions 104, followed by application of, among other possible layers, a temporary barrier layer 106 which provides for delayed release of the therapeutic agent from the therapeutic-agent-containing regions 104. A similar effect can be obtained as shown in FIG. 7 by applying coated particles that include a therapeutic-agent-containing region 104 in the form of a core and a temporary barrier layer 106 in the form of a coating/shell to a substrate 102. Cores 104 may be provided in various regular and irregular shapes including spherical shapes and non-spherical shapes (e.g., rectangular solids, etc.). Coated cores 104/106 may be provided in a variety of sizes, ranging for example, from 2 to 100 μm in diameter (for a sphere) or length (for a non-sphere), among other sizes. The temporary barrier layers 106 (i.e., shells) for such cores for such cores 104 can be, for example, organic or inorganic in nature.

For example, cores of pure drug or drug combined with a biodisintegrable or biostable organic or inorganic matrix material can be coated using a suitable process such as physical vapor deposition (PVD), chemical vapor deposition (CVD) or atomic layer deposition (ALD).

As a specific example, therapeutic-agent-containing cores may be coated with inorganic shells using ALD, which can be performed in conjunction with a fluidized bed reactor to provide improved gas/particle contact and thermal efficiency. For example, X. Liang et al., J. Am. Ceram. Soc., 90(1), 2007, 57-63, describe a process whereby alumina is deposited on the surface of high-density polyethylene particles by repeated exposure to trimethylaluminum and H₂O vapor at 77° C. in a repeated alternating sequence. Id. Analogous processing may be employed to coat drug-containing particles with various metals and non-metals other than alumina at relatively low temperatures, including Ta₂O₅ (see, e.g., M. Seman et al, Applied Physics Letters 90, 131504 (2007) who describe plasma-enhanced chemical vapor deposition of Ta₂O₅ at temperatures as low as 90° C. from pentaethoxy tantalum and O₂), TiO₂/titania (see, e.g., Jae-Hwang Lee et al., Applied Physics Letters 90 151101 (2007) who describe ALD deposition of TiO₂ at 100° C. from TiCl₄ and H₂O) and SiO₂/silica (see, e.g., J. W. Klaus et al., Surface Review and Letters, Vol. 6, Nos. 3 & 4 (1999) 435-448, who describe ALD deposition of SiO₂ at temperatures as low as 300K from SiCl₄ and H₂O using pyridine as a catalyst; see also U.S. Pat. No. 7,077,904 to Cho et al.), among many others, for use in the invention.

A specific example of a technique for forming therapeutic-agent-containing regions with organic shells that act as temporary barrier layers is layer-by-layer (LbL) polyelectrolyte deposition.

In some embodiments, a system is chosen whereby therapeutic agent is released due to biodegradation of the organic shell. Pub No. US 2005/0129727 to Weber et al. describes capsules that comprise (a) a drug-containing core and (b) a biodegradable polyelectrolyte multilayer encapsulating the drug-containing core.

In other embodiments, a system is chosen whereby therapeutic agent is released due to rupture of the organic shell. As a specific example of a technique for forming therapeutic-agent-containing regions with rupturable organic shells that act as temporary barrier layers, Bruno G. De Geest et al., Adv. Mater., 17 (2005) 2357-2361 describe a process whereby biodisintegrable dextran-based microgels (gel microspheres) formed by the copolymerization of dextran-hydroxyethyl methacrylate with diethylaminoethyl methacrylate (dex-HEMA-DMAEMA) to created positively charged crosslinked microspheres. The microspheres were encapsulating with multiple alternating layers of positively and negatively charged polyelectrolytes, specifically poly(allylamine hydrochloride) (PAH) and poly(sodium 4-styrenesulfonate) (PSS). The coating was found to be permeable to 20 kDa FTC-dextran at pH 7 and impermeable at pH 9. As a result, the major degradation product of the dex-HEMA-DMAEMA (19 kDa dextran) were expected to remain in the capsules at pH 9, resulting in an increase in internal osmotic pressure and rupture. Rupture was observed after 105 minutes. Further work done by De Geest (Ph.D. Thesis entitled “Polyelectrolyte microcapsules for pharmaceutical applications” presented 5 Dec. 2006 at the Faculty of Pharmaceutical Sciences, University of Gent, Belgium) reveals that increasing the number of PAH/PSS bilayer pairs to 6 made the layers also impermeable at pH 7. Also Bruno De Geest, Journal of Controlled Release 135 (2009) 268-273, reveals that using dextran sulfate and poly-L-arginine is suitable for applications at around pH 7.4, which is the pH of the blood. The time it takes for the capsules to rupture can be programmed by controlling the build up of the osmotic pressure in the capsules, which is controlled by both the concentration of the dex-HEMA-DMAEMA in the capsules as well as the crosslink density. Furthermore, the maximum pressure a capsule can withstand is given by Laplace's law, which states that the maximum pressure a capsule can withstand is inversely dependent on the radius of the capsule.

As another example of organic particles with time-delay release, see Crommelin et al. Journal of Controlled Release 87 (2003) 81-88, who describe release time delays of up to two weeks at conditions of pH 7 and 37° C. for protein loaded degradable hydroxyethyl methacrylated dextran (dex-HEMA) hydrogel microspheres.

Although the substrate in FIG. 7 is shown as smooth, in other embodiments (not shown) the particles may be disposed within the pores of a porous surface. For example, porous inorganic layers may be created by first forming inorganic-polymer hybrid structures, after which the polymeric material is subsequently removed, for example, by thermal and/or chemical processes (e.g., by burning out the polymer or by exposing the polymer to a solvent), leaving behind porous ceramic structures. See, e.g., US 2008/0188836 to Weber et al. For instance, porous ceramic structures may be created using sol-gel based techniques, including sol-gel based techniques wherein sol-gel/polymer hybrid structures are formed and the polymeric phase (which may be of various shapes and sizes, including, microspheres, etc.) are removed by thermal decomposition to yield a porous ceramic structure. See, e.g., Pub No. US 2009/0029077 to Atanasoska et al. and Pub No. US 2008/0051881 to Feng et al.

If desired, a layer of material 106a can be provided on the substrate 102 after applying the coated cores 104/106 as shown in FIG. 8. For example, in the case of inorganic coated particles (e.g., particles with a coating formed by ALD), a compatible material layer 106a may be formed of the same material as the inorganic shells 106 of the particles. As another example, in the case of organic coated particles (e.g., particles with a coating formed by an LbL process), a compatible material layer 106a may be formed from one or more polyelectrolyte sub-layers. As with FIG. 7, although the substrate in FIG. 8 is shown as smooth, in other embodiments (not shown) the particles may be disposed within the pores of a porous surface.

Similarly, if desired, a layer of material 106b can be provided on the substrate 102 prior to applying the coated particles as shown in FIG. 9. Analogous to the preceding paragraph, in the case of inorganic coated particles (e.g., particles with a coating formed by ALD), a compatible material layer 106b may be formed of the same material as the inorganic shells 106 of the particles. As another example, in the case of organic coated particles (e.g., particles with a coating formed by an LbL process), a compatible material layer 106b may be formed from one or more polyelectrolyte sub-layers (e.g., where the outermost polyelectrolyte sub-layer of layer 106b is opposite in sign from an outermost polyelectrolyte sub-layer of the polyelectrolyte shell 106 of the particles 104/106, thereby creating an electrostatic attraction).

A further layer 106a like that shown in FIG. 8 may be provided in the structure of FIG. 9 to create a structure like that show in FIG. 9A.

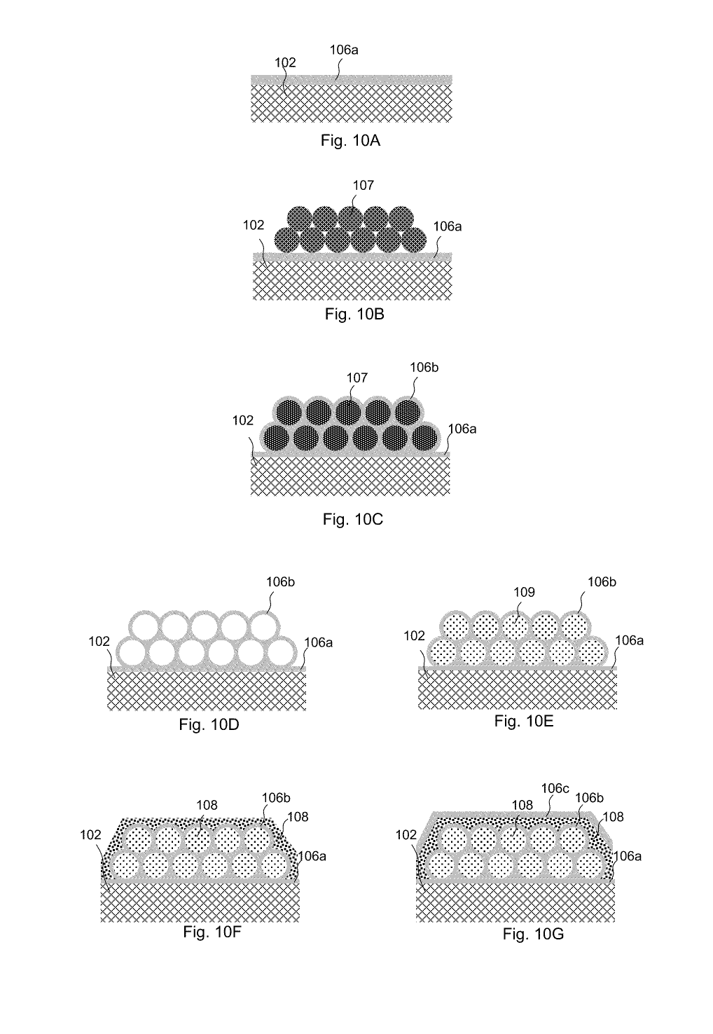

Other, more complex schemes can be employed to form structures in accordance with the invention as shown in FIGS. 10A-10G. For example, referring now to FIG. 10A, in a first step, a substrate 102 can be coated with a temporary barrier layer material 106a (e.g., an inorganic layer formed using ALD). Then, a collection of polymer microspheres is applied over the barrier layer material 106a as shown in FIG. 10B. The microspheres may range, for example, from 4 to 100 μm in diameter and are preferably monodisperse (i.e., of substantially the same size) so as to allow for a regular particle array (e.g. a face-centered-cubic array). In a subsequent step, an additional layer of temporary barrier material 106b is formed over the coated substrate and particle array (e.g., using ALD) to form a structure like that shown in FIG. 10C. The structure may then be heated to a temperature that is sufficient to vaporize the polymer microsphere material, forming an ordered face-centered-cubic array of interconnected air-filled spheres in a matrix of barrier material 106a,106b as shown in FIG. 10D.

The ability to make similar structures has been demonstrated in M. Scharrer, Applied Physics Letters 86, 151113 (2005) who describe a process whereby polystyrene microspheres are first deposited to form a multilayer structure. This structure is then infiltrated with ZnO using a low-temperature atomic layer deposition process using diethyl zinc and H₂O as precursor gases, after which the polystyrene microspheres are removed by heating the structures in air to 550° C. for 30 min. to create an ordered face-centered-cubic array of interconnected, spherical air-filled holes in the ZnO matrix.

As another example of a method of forming a structure like that of FIG. 10D, ALD-coated polymeric particles (e.g., alumina-coated polyethylene particles as described in Liang et al., supra) can be heated in air at elevated temperature to burn out the polymeric material and create hollow ceramic particles. Such particles can subsequently be applied to a substrate to provide a structure like that of FIG. 10D.

The holes/pores of the structure of FIG. 10D can then be at least partially filled with drug 109 (e.g., by immersion in a drug solution, by applying a spray or drops of a drug solution, etc.) to provide a structure like that shown in FIG. 10E. A porous layer 108 can then be applied to the structure, e.g., using a low temperature process such as the Mantis process described above, to create a structure like that shown in FIG. 10F. Finally, to provide a structure which is capable of delayed release, an additional layer of temporary barrier material 106c is applied as shown in FIG. 10G.

As indicated above, a wide variety of therapeutic agents can be employed in conjunction with the medical devices of the present invention including those used for the treatment of a wide variety of diseases and conditions (i.e., the prevention of a disease or condition, the reduction or elimination of symptoms associated with a disease or condition, or the substantial or complete elimination of a disease or condition). Therapeutic agents include non-genetic therapeutic agents, genetic therapeutic agents, and cells. Therapeutic agents may be used singly or in combination.

Exemplary therapeutic agents for use in connection with the present invention include: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists, including PPAR-alpha, gamma and delta agonists, such as rosiglitazone, pioglitazone, netoglitazone, fenofibrate, bexaotene, metaglidasen, rivoglitazone and tesaglitazar, (bb) prostaglandin E agonists, including PGE2 agonists, such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, (ee) thymosin beta 4, (ff) phospholipids including phosphorylcholine, phosphatidylinositol and phosphatidylcholine, (gg) VLA-4 antagonists and VCAM-1 antagonists, (hh) iron chelating agents including siderophores such as hydroxamates, ethylenediamine tetra-acetic acid (EDTA) and its analogs, and catechols.

Therapeutic agents also include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, biolimus, Epo D, dexamethasone, estradiol, halofuginone, cilostazole, geldanamycin, alagebrium chloride (ALT-711), ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Numerous therapeutic agents, not necessarily exclusive of those listed above, have been identified as candidates for vascular treatment regimens, for example, as agents targeting restenosis (antirestenotics). Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists such as bosentan, sitaxsentan sodium, atrasentan, endonentan, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, atorvastatin, fluvastatin, simvastatin and cerivastatin, (u) fish oils and omega-3-fatty acids, (v) free-radical scavengers/antioxidants such as probucol, vitamins C and E, ebselen, trans-retinoic acid, SOD (orgotein) and SOD mimics, verteporfin, rostaporfin, AGI 1067, and M 40419, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) matrix metalloprotease (MMP) pathway inhibitors such as marimastat, ilomastat, metastat, batimastat, pentosan polysulfate, rebimastat, incyclinide, apratastat, PG 116800, RO 1130830 or ABT 518, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, and epothilone), caspase activators, proteasome inhibitors, angiogenesis inhibitors (e.g., endostatin, angiostatin and squalamine), sirolimus, everolimus, biolimus tacrolimus, zotarolimus, cerivastatin, flavopiridol and suramin, (aa) matrix deposition/organization pathway inhibitors such as halofuginone or other quinazolinone derivatives, pirfenidone and tranilast, (bb) endothelialization facilitators such as VEGF and RGD peptide, (cc) blood rheology modulators such as pentoxifylline and (dd) glucose cross-link breakers such as alagebrium chloride (ALT-711).

Numerous additional therapeutic agents useful for the practice of the present invention are also disclosed in U.S. Pat. No. 5,733,925 to Kunz, the entire disclosure of which is incorporated by reference.

### Example

An example will now be described in which a stent is provided with a delayed release coating, in accordance with an embodiment of the invention. In a first step, a stainless steel stent is cleaned with alcohol. This is followed by deposition of an initial ALD layer on the stent to avoid any open spots on the stent where the polystyrene spheres (see below) are in initial contact with the stent. Conformal deposition of TiO₂ is performed on the stent by ALD at 100° C. A 5 nm thick TiO₂ layer is deposited in 60 cycles of deposition, where each cycle consists of TiCl₄ injection for 0.5 s, N₂ purge for 10 s, H₂O injection for 10 s, and N₂ purge for 30 s. Such a procedure is described in Jae-Hwang Lee et al., Applied Physics Letters 90 151101 (2007).

After TiO₂ deposition, polystyrene microspheres in water are deposited on the stent struts. The process is performed at 70° C. in an oven whereby drops are deposited on the stent structure, depositing the drops on the upper (horizontal) row of struts, with 100 micrometer spacing between drops, allowing 30 seconds before the stent is rotated to coat the next row of struts with drops, thereby creating distinct regions of polystyrene spheres, separated by regions without spheres. A Microdrop ADK-501 (Microdrop Technologies GmbH, Muehlenweg 143, D-22844 Norderstedt, Germany) autodispensing system with a 30 micrometer nozzle dispensing 25 pL per drop is used for this purpose. Polystyrene microspheres are purchased from Microparticles, Berlin, Germany, PS-R-0.35 1% solution, average diameter 330 nm. The stent is dried at 70° C. for an hour.

The ALD process is used to fill the spaces between the polystyrene microspheres. The same process is used as above, but now with sufficient cycles (e.g., ˜800) to fill the inner spaces between the spheres.

The polystyrene sphere templates are then removed by repeated heating to 300° C. under vacuum for 3 hours followed by rinsing in acetone, thereby creating a surface with distinct porous regions of interconnected spherical pores.

Drug solution is then dispensed into the resulting porous structure dropwise using the Microdrop ADK-501 autodispensing system described above with stent rotation. The solution is paclitaxel dissolved in tetrahydrofuran (THF). For each porous region, an initial 5 drops of 20 pL having a 5% paclitaxel concentration are applied, followed by 10 drops of 20 pL having a 1% paclitaxel concentration. 5 seconds in between each dispensing step is employed to allow solvent to flash off.

The preceding step is followed by a quick rinsing step in which the stent is immersed in THF for 5 seconds to remove any surface drug remaining, thereby exposing part of the porous TiO₂ structure.

Tantalum (Ta) nanoparticles are then deposited to a Ta layer thickness of 55 nm. Ta nanoparticles of 10 nm average diameter are deposited at 1100 V using the Nanogen 50 nanocluster depositon system from Mantis Deposition Ltd., thereby creating a nanoporous outer Ta film.

In a final step, a 10 nm thick alumina biodisintegrable temporary barrier coating is provided on the structure using the ALD process. The alumina layer is deposited in 100 cycles of deposition, where each cycle consists of trimethylaluminum injection for 5 s, N₂ purge for 5 s, H₂O injection for 5 s, and N₂ purge for 5 s.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising (a) a substrate, (b) a plurality of therapeutic-agent-containing regions comprising a therapeutic agent disposed over said substrate and spaced laterally relative to one another, and (c) an inorganic biodisintegrable temporary barrier layer disposed over said substrate and said therapeutic-agent-containing regions such that release from said therapeutic-agent-containing regions is delayed for a period of 5 to 28 days after implantation or insertion of said device in the body of a subject.

2. The implantable or insertable medical device of claim 1, wherein the inorganic biodisintegrable temporary barrier layer is vapor deposited.

3. The implantable or insertable medical device of claim 2, wherein the inorganic biodisintegrable temporary barrier layer is deposited by atomic layer deposition.

4. The implantable or insertable medical device of claim 2, wherein the inorganic biodisintegrable temporary barrier layer comprises a material selected from alumina, silica, hydroxyapatite, and magnesium fluoride.

5. The implantable or insertable medical device of claim 1, wherein an additional layer of inorganic biodisintegrable material is disposed between said therapeutic-agent-containing regions and said substrate.

6. The implantable or insertable medical device of claim 1, further comprising a porous layer.

7. The implantable or insertable medical device of claim 7, wherein said therapeutic-agent-containing regions are formed within said porous layer and wherein said inorganic biodisintegrable temporary barrier layer is disposed over said porous layer.

8. The implantable or insertable medical device of claim 6, wherein said porous layer is disposed on said therapeutic-agent-containing regions and on bare portions of said substrate between said therapeutic-agent-containing regions and wherein said inorganic biodisintegrable temporary barrier layer disposed on said porous layer.

9. The implantable or insertable medical device of claim 1, wherein said therapeutic-agent-containing regions comprise inorganic shells that are at least partially filled with a therapeutic agent.

10. The implantable or insertable medical device of claim 9, wherein said shells are atomic layer deposited shells.

11. The implantable or insertable medical device of claim 6, wherein said porous layer is disposed over said substrate, wherein said therapeutic-agent-containing regions are disposed over said porous layer, and wherein said inorganic biodisintegrable temporary barrier layer is disposed over said therapeutic-agent-containing regions.

12. The implantable or insertable medical device of claim 6, wherein said inorganic biodisintegrable temporary barrier layer is disposed on said therapeutic-agent-containing regions and bare portions of said substrate between said therapeutic-agent-containing regions, and wherein said porous layer is disposed on said inorganic biodisintegrable temporary barrier layer.

13. The implantable or insertable medical device of claim 6, wherein said porous layer is disposed on a face of said substrate that is opposite that of the biodisintegrable temporary barrier layer.

14. The implantable or insertable medical device of claim 1, wherein said therapeutic-agent-containing regions correspond to a core of therapeutic-agent-containing material within a shell, which therapeutic-agent-containing regions are disposed between said substrate and said inorganic biodisintegrable temporary barrier layer.

15. The implantable or insertable medical device of claim 14, wherein said shell is formed from the same inorganic biodisintegrable material as said inorganic biodisintegrable temporary barrier layer.

16. The implantable or insertable medical device of claim 1, wherein said medical device is a stent.

17. The implantable or insertable medical device of claim 16, wherein said therapeutic-agent-containing regions are disposed over the abluminal surface of said stent but not over the luminal surface.

18. The implantable or insertable medical device of claim 16, wherein said therapeutic-agent-containing regions comprise an antirestenotic agent.

19. The implantable medical device of claim 1, wherein an organic biodegradable temporary barrier layer is deposited by spray coating, roll coating, ink jet droplet deposition, micro-contact printing, nanopipetting, or dip pen nanolithography.

20. An implantable or insertable medical device comprising (a) a substrate, (b) a plurality of capsules disposed over the substrate, each of said capsules comprising a therapeutic-agent-containing core region and a polyelectrolyte multilayer shell surrounding said core region, said capsules being adapted to rupture during a period of ranging from 5 to 28 days after implantation or insertion of said device in the body of a subject.

21. The implantable or insertable medical device of claim 20, further comprising a porous layer disposed over said capsules.
